(19) United States
(12) Patent Application Publication
(10) Pub. No.: US 2003/0135102 A1
(43) Pub. Date: Jul. 17, 2003

(54) Method and system for registration and guidance of intravascular treatment

(76) Inventors: Everette C. Burdette, Champaign, IL (US); Dana L. Deardorff, Oakland, CA (US)

Correspondence Address: THOMPSON COBURN, LLP, ONE FIRSTAR PLAZA, SUITE 3500, ST LOUIS, MO, 63101, US

(21) Appl. No.: 10/310,565

(22) Filed: Dec. 5, 2002

Related U.S. Application Data
(63) Continuation-in-part of application No. 09/573,415, filed on May 18, 2000, now Pat. No. 6,512,942.
(63) Continuation-in-part of application No. 09/897,326, filed on Jul. 2, 2001.
(60) Provisional application No. 60/337,932, filed on Dec. 5, 2001.

(51) Int. Cl. A61B 5/05

(52) U.S. Cl. 600/407

(57) ABSTRACT

A system and method for intravascular treatment planning and therapy on a patient. The system includes a structure for holding a treatment source, such as a radiation source, and also moving the treatment source through a patient's vascular system using a positioning device which includes a component to establish the location relative to a particular reference position. An imaging device is also disposed in association with a vascular treatment area and the treatment source to register the resulting image information, enabling formation of three dimensional image in real time for use by a clinician.

### CROSS REFERENCE AND PRIORITY CLAIM TO RELATED APPLICATION

This application claims the benefit under 35 U.S.C. § 119(e) of provisional patent application Ser. No. 60/337,932 entitled “System for Registration and Guidance of Intravascular Treatment”, filed Dec. 5, 2001, the entire disclosure of which is incorporated herein by reference.

This application is also a continuation-in-part of pending U.S. application Ser. No. 09/573,415 filed May 18, 2000 entitled Real Time Brachytherapy Spatial Registration and Visualization System, the entire disclosure of which is incorporated herein by reference.

This application is also a continuation-in-part of pending U.S. application Ser. No. 09/897,326 filed Jul. 2, 2001 entitled Virtual Reality 3D Visualization for Surgical Procedures, the entire disclosure of which is incorporated herein by reference.

### FIELD OF THE INVENTION

The present invention relates in general to a system and method for treatment of intravascular disease and other portions of the human body. More particularly, the invention is directed to a system and method for spatial registration of an intravascular treatment device and for brachytherapy of intravascular tissue.

### BACKGROUND OF THE INVENTION

Coronary artery disease and other vascular diseases are a serious health problem throughout the world. In the U.S. alone more then ten million Americans are newly affected each year. Approximately one to two million of these patients will undergo balloon angioplasty, and a substantial fraction of these patients will require placement of a vascular stent to open an affected arterial vessel. However, a significant percentage of those treated by angioplasty (about 30-50%) will experience restenosis of the arterial vessel within a relatively short period of time (about 6-12 months).

Transluminal intravascular brachytherapy is one treatment approach that has shown potential to prevent restenosis of the coronary arteries following angioplasty and stent procedures. This brachytherapy procedure is performed using intravascular ultrasound (IVUS) imaging for visualization of the stenosed artery. However, this procedure is currently performed “blind” without any quantitative spatial registration of the radiation source to the ultrasound images, as well as without any calculated dosimetry of the radiation treatment. Because of the nature of this “open-loop” procedure, clinical results with vascular brachytherapy have been highly variable.

### SUMMARY OF THE INVENTION

It is therefore an object of the invention to provide an improved system and method for intravascular treatment planning and therapy.

It is another object of the invention to provide an improved method and system for treatment of a variety of portions of the human body.

It is also an object of the invention to provide an improved system and method for intravascular brachytherapy treatment planning and therapy.

It is an additional object of the invention to provide an improved system and method for spatial registration and positioning of an intravascular treatment device relative to intravascular tissue undergoing therapy.

It is a further object of the invention to provide an improved system and method for guidance of an intravascular treatment device.

It is still another object of the invention to provide an improved system and method for control and registration of catheter devices for containment and precise positioning of intravascular treatment.

It is yet a further object of the invention to provide an improved system and method for control of radiation sources for brachytherapy intravascular treatment.

It is also an additional object of the invention to provide an improved system and method for substantially real time imaging of intravascular tissue and treatment.

It is yet another object of the invention to provide an improved system and method for providing substantially real time guidance and dosimetry feedback during intravascular treatment.

It is yet another object of the invention to provide an improved system and method for digitized control of positioning of an intravascular treatment device within a vascular portion of a patient's body.

These and other objects and advantages of the invention will become apparent from the following specification and claims taken with the drawings described hereinbelow.

In an effort to achieve these objectives, disclosed herein is a system for spatial registration of an intravascular treatment source used for therapy on a patient, the system comprising: (a) a treatment structure for holding a treatment source, both of which are moveable, through a vascular system; (b) a positioning device coupled to the treatment structure, said device being configured to enable establishing spatial position of the treatment structure; and (c) an imaging device disposed to view a treatment area of the patient to provide image data for display of the treatment source disposed in the patient.

Also disclosed herein is a method of intravascular treatment of a patient, the method comprising the steps of: (a) providing a treatment energy source coupled to a positioning component; (b) positioning the treatment energy course in a vascular system using the positioning component which includes a coupling to a position sensor, thereby establishing a distance of travel of the treatment energy source in the vascular system; (c) registering image information characteristic of the treatment energy source to image information characteristic of at least a portion of the vascular system of the patient; and (d) generating, in substantially real time, three dimensional registered image information for the treatment energy source, and the vascular system of the patient.

These and other features of the invention will be in part pointed out and in part apparent upon review of the following description and attached figures.

### BRIEF DESCRIPTION OF THE DRAWINGS

FIG. 1 illustrates a functional block diagram of an embodiment of the invention; and

FIG. 2 illustrates the preferred embodiment of the present invention in operation.

### DETAILED DESCRIPTION OF THE PREFERRED EMBODIMENT

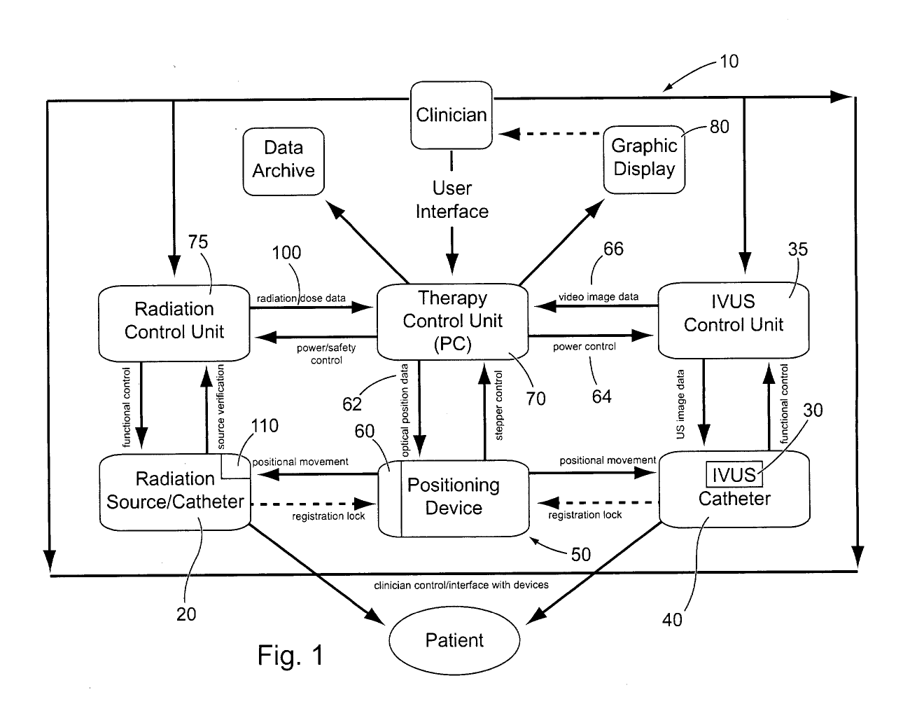

A preferred embodiment of the invention is illustrated schematically as system 10 in the functional block diagram of FIG. 1. This system 10 is directed to providing therapeutic treatment to a portion of the body of a patient, including establishing the spatial position of an intravascular treatment source 20 in an intravascular portion of the body of a patient. In order to achieve an advantageous treatment of vascular disease (and other diseases), the treatment source 20 should be precisely positioned within a system of the patient, such as a vascular system. Most preferably, the treatment source 20 should be spatially registered relative to a known position in order to achieve the most desired location relative to a treatment site. The treatment source may be spatially registered relative to any reference position, such as for example, a portion of the patient's body, a fixture being used to move the treatment source, and an imaging device 30. The preferred reference point for the image is the distal location of the diseased region (plaque, lesion) of the vessel to undergo imaging and treatment. The reference point for the catheter positioning (for either the treatment source or the imaging transducer) is a reference mark on the catheter that is a fixed distance from the distal end of the catheter and is referenced to the positioning device/fixture.

The imaging device 30 can be, for example, an intravascular ultrasound probe (IVUS), an MRI unit, an X-ray unit or any conventional device able to provide the necessary imaging information for the clinician treating the patient. This imaging device 30 is therefore used to establish the location of the treatment source 20 and portions of the patient's body, relative to the reference position and also to portions of the human body (as determined from features depicted in the image data). The imaging device 30 can be positioned by a control device 35 which will be described hereinafter.

The treatment source 20 most preferably is a radioactive source, such as any commercially available radioactive medium, such as seeds, wires, or liquids. Additionally, the treatment source 20 can be a microwave source, ultrasound source or other such source that applies therapeutic energy to the patient. The treatment source 20 is positioned intravascularly. Preferably, a treatment structure holds the treatment source. The treatment structure may be a container, such as a catheter 40 (in a most preferred embodiment). However, any conventional device that can be used to transport the treatment source 20 either manually or automatically through a portion of the patient's body may be used.

Guidance of the treatment source catheter 40 preferably uses a positioning device 50 which comprises a physical coupling. The positioning device preferably uses guidewires, fibers, or other connective leads to move the treatment source 20 and the catheter 40 coupled thereto through the patient's body. The positioning device 50 also can further include a positioning drive, such as a digital or analog drive 60 which allows precise travel and tracking and/or positional encoding of the treatment source 20's position within the patient.

The positional encoding is preferably accomplished by a positional encoding system comprised of computer software executed by therapy control unit (TCU) 70 to utilize positional data 62 received from the drive 60. Once positional encoding is achieved, the TCU 70 can provide a power control signal 64 to the imaging control device 35 which activates and operates the imaging function of the imaging device 35.

In a most preferred embodiment, both the treatment source 20 and the imaging device 30 can be positioned by a common positioning device 50 under control of the software in the therapy control unit 70, such as the control device 35. In one form of the invention, the image device catheter 40 can include a channel for transporting any conventional therapeutic medication, including molecular and genetic therapeutic targeting, to the vascular tissue.

The system 10 further includes a therapy control unit, such as computer 70 and radiation control unit 75. The TCU 70 executes computer software to operate the control device 35 and to process image data 66 received from the imaging device 30. The processed data is then output as an image on graphic display 80. Throughout the system 10, various connections are depicted, such as a connection between the computer 70 and the control device 35 and a connection between the computer 70 and the treatment source 20. These various connections can be any conventional data communication or electrical coupling device, including but not limited to hard wire connections or electromagnetic wave communication devices.

The resulting images provided to the clinician are spatially registered by computer software executed on the computer 70. This spatial registration enables viewing the location of the treatment source 20 relative to the vascular treatment region of interest. This spatial registration feature also permits reproducibility of positioning for repetitive or other treatment protocols which require a subsequent return to a particular vascular treatment region. In addition, the image data sent from control unit 35 to TCU 70 and shown on the graphic display 80 can include spatial registration of a sequence of two dimensional slices taken along the intravascular pathway, thereby enabling construction of three dimensional images. Further by virtue of the rapidity with which such images can be formed, the clinician can be provided with substantially real time three dimensional images for viewing on the graphic display 80. Thus, the therapeutic treatment can be observed in virtually real time, thereby enabling optimized clinician action for administering treatment to the patient.

In addition, the image information can be manipulated for evaluation by the computer 70 by executing image data to rotate images, translate along selected directions, change image appearance or translucency, and can be further examined subsequent to treatment for evaluating effectiveness of the therapeutic procedure.

The computer 70 can further analyze treatment source data 100 output by a conventional radiation or energy sensor 110 that is disposed near the vascular tissue being treated via the radiation control unit 75. Alternately, the treatment source data 100 can arise from a calculation based on the known energy field from the treatment source 20. The data 100 is characteristic of the radiation dose or other energy being deposited in vascular tissue and nearby regions of the patient's body. This data 100 is thus analyzed by virtue of conventional computer software such as the interplant® brachytherapy treatment planning and guidance system (available from Computerized Medical Systems, Inc. of St. Louis, Mo.; the details of which are disclosed in U.S. Pat. No. 6,129,670 and pending U.S. application Ser. No. 09/573,415, the disclosures of both of which are incorporated herein by reference) executed by the computer 70. The resulting radiation dose or energy deposition information can be spatially registered to the position of the source and the surrounding region of the patient's body via the positioning device 50, and then be output for viewing on the graphic display 80 by the clinician. The energy deposition information can also be displayed in substantially real time, as is the imaging information 45 descriptive of the spatial registration of the treatment source 20, the catheter 40 and portions of the patient's body. The clinician can then choose to move the treatment source 20, change the time period of energy or radiation treatment, change the strength or activity of the source, or even insert additional ones of the radiation source 20 (such as radioactive seeds) to achieve the desired dosage.

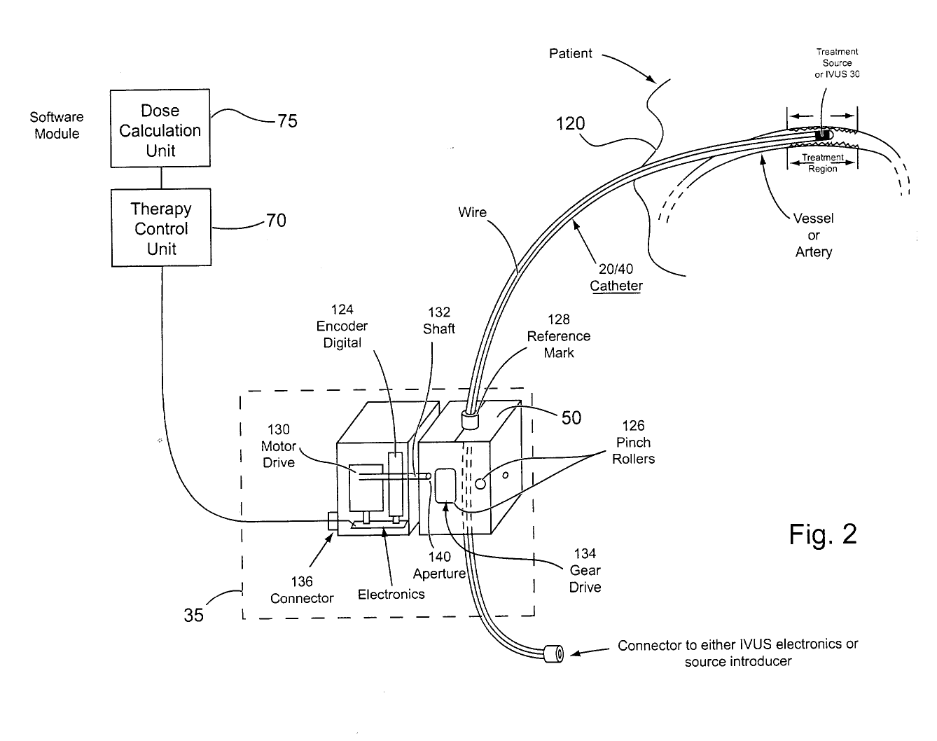

FIG. 2 depicts the preferred embodiment of the present invention in operation. That figure shows the positioning device digital encoder 124 connected to the control unit 35 for readout of the position data for either the image device catheter 40, the treatment source catheter 20, or an alternative catheter containing an imaging device with a working channel for treatment. Preferably, the imaging device 30 is an IVUS. The catheter 20/40 is placed within the positioning device 50.

The positioning device 50 contains pinch rollers 126 that grasp the catheter 20/40 at an initialization location along the catheter length which is marked on the catheter by reference mark 128. The distance from the mark 128 on the catheter to the treatment source position and/or IVUS imaging transducer position within the catheter is known apriori. The positioning device 50 is sterile and has an aperture 140 which receives a drive shaft 132 from a separate drive unit 130.

The drive unit 130 contains a motorized or manual gear drive mechanism 134 which drives the shaft 132 that is inserted into the positioning device 50. The drive unit 130 can be controlled by the TCU 70. The digital encoder 124 is connected to the drive shaft 132 and digitally encodes the shaft rotation, wherein the catheter position is determinable from this shaft rotation. A preferred digital encoder for the present invention is the DS04DD01 manufactured by Netzer Precision. This encoded positional information is sent electronically to the TCU 70 via connector 138. The TCU 70 interfaces with computer software within the Radiation Control Unit 75 which computes the dosimetry for the treatment source.

As noted above, the radiation source may be x-radiation (beta or gamma), microwave, ultrasound, or some other therapeutic source that is localized near the end of the catheter that is inserted into the vessel and placed at the treatment position under image visualization. Visualization can be standard fluoroscopy used for guidance of vascular procedures, MRI imaging, or IVUS ultrasound imaging.

The image information is initially correlated with the position of the catheter in the target vessel and the position of the catheter in the positioning device 50 (the catheter's position in the position device being determinable from the known distance of the source to the reference mark 128 and from the shaft information logged by the digital encoder 124 and TCU 70). The image data is continually sent from the imaging device, typically IVUS, to the Therapy Control Unit. The position of the IVUS imaging transducer is known by the Therapy Control Unit from the data provided by the positioning device 50 and digital encoder 124. Each image from the imaging device (IVUS) is spatially registered with the digitally encoded imaging transducer position. This is accomplished by registering the images with respect to each other using the encoded position for each image. The software, then computes the dose distribution in the space described by the images using software based upon the American Association of Physicists in Medicine Task Group No. 43 standard. The software is preferably as described by the inventor is U.S. Pat. Nos. 6,129,670 and 6,256,529 (the disclosure of which is incorporated herein by reference), and pending U.S. application Ser. Nos. 09/573,415 and 09/897,326 (both of which are incorporated by reference above). A similar method is used to localize a separate treatment source based upon its digitally encoded movement within the positioning device relative to its initialization position and visualization image information. The Therapy Control Unit correlates the digitally encoded positional information of the Treatment Source or IVUS transducer to its location within the vessel. The initial referencing mark 128 on the catheter and knowledge of position relative to the reference as well as the image make registration of the position of the source/transducer within the vessel possible. The spatial registration information is used to update source location to software that computes and continuously updates the delivered dosimetry to the vessel as a function of position of source within the vessel and as a function of time.

While preferred embodiments of the invention have been shown and described, it will be clear to those skilled in the art that various changes and modifications can be made without departing from the invention in its broader aspects as set forth in the claims provided hereinafter.

What is claimed is:

1. A system for spatial registration of an intravascular treatment source used for therapy on a patient comprising:
a treatment structure for holding a treatment source, both of which are moveable through a vascular system;
a positioning device coupled to the treatment structure, said device being configured to enable establishing spatial position of the structure; and
an imaging device disposed to view a treatment area of the patient to provide image data for display of the treatment source disposed in the patient.

2. The system as defined in claim 1 wherein the treatment structure comprises an intravascular catheter.

3. The system as defined in claim 2 wherein the treatment structure includes connections selected from the group of guidewires, fibers and connective leads coupled thereto.

4. The system as defined in claim 1 wherein the imaging device is coupled to the positioning device.

5. The system as defined in claim 1 where the imaging device is coupled to the treatment structure.

6. The system as defined in claim 1 wherein the positioning component further comprises at least one of a digital drive and a mechanical/analog drive.

7. The system as defined in claim 6 wherein the positioning component further comprises a positional encoding system.

8. The system as defined in claim 1 wherein the positioning component communicates the encoded spatial position of at least one of the imaging source and the treatment source to a common control unit.

9. The system as defined in claim 1 wherein the imaging device comprises an ultrasound device.

10. The system as defined in claim 9 further including a computer and executable computer software for processing the image data to generate a graphic display of at least one of the treatment source when present in the vascular system and the treatment structure.

11. The system as defined in claim 10 further including computer software for registering two dimensional image data to provide three dimensional images, as well as manipulating or transforming two dimensional or three dimensional appearance, size and shape.

12. The system as defined in claim 1 further including a control device and an electrical communication between the positioning device and the control device.

13. The system as defined in claim 12 wherein the electrical communication comprises at least one of an electrical coupling and an electromagnetic communications system.

14. The system as defined in claim 1 wherein the positioning component provides spatial registration data in association with the image data to generate a spatially registered image.

15. The system as defined in claim 9 wherein the spatially registered image comprises at least one of at least one two dimensional image slice and a three dimensional image generated in substantially real time.

16. The system as defined in claim 1 further including means for calculating the three dimensional radiation dose distribution arising from the treatment source and outputting dosage data for display.

17. The system as defined in claim 16 wherein the radiation dosage is spatially registered with the treatment source disposed in the patient.

18. The system as defined in claim 17 where the spatially registered radiation dosage is displayed graphically in three dimensions and in substantially real time.

19. The system as defined in claim 1 wherein the imaging device comprises an ultrasound transducer disposed within the vascular system.

20. The system as defined in claim 1 further including a catheter for delivering at least one of medication to the patient and therapeutic ultrasound energy to the patient.

21. The system as defined in claim 1 wherein the treatment sources comprises at least one of a radioactive source, a microwave source, a radiofrequency source, a laser source, an ionizing radiation source, and an ultrasound energy source.

22. The system as defined in claim 1 wherein the positioning device is movable through the vascular system.

23. A method of intravascular treatment of a patient, comprising the steps of:
providing a treatment energy source coupled to a positioning component;
positioning the treatment energy source in a vascular system using the positioning component which includes a coupling to a position sensor, thereby establishing a distance of travel of the treatment energy source in the vascular system;
registering image information characteristic of the treatment energy source to image information characteristic of at least a portion of the vascular system of the patient; and
generating in substantially real time, three dimensional registered image information for the treatment energy source and the vascular system of the patient.

24. The method as defined in claim 23 further including the step of registering image information characteristic of an energy source container to at least one of the image information from the treatment energy source and the image information from the vascular system of the patient.

25. The method as defined in claim 23 wherein the treatment energy source further includes an ultrasound imaging source to form the image information.

26. The method as defined in claim 15 wherein the treatment energy source is selected from the group consisting of at least one radioactive seed, an ultrasound energy source, an ionizing radiation source, a microwave energy source and a laser.

27. The method as defined in claim 26 further including the step of applying energy to a portion of the vascular system of the patient, determining dosage of energy and displaying the energy dosage in registration with the treatment energy source and the portion of the vascular system.
